(19) United States
(12) Patent Application Publication
(10) Pub. No.: US 2003/0153901 A1
(43) Pub. Date: Aug. 14, 2003

(54) Drug delivery panel

(75) Inventors: Steve Herweck, Nashua, NH (US); Paul Martakos, Pelham, NH (US); Theodore Karwoski, Hollis, NH (US); Roger Labrecque, Londonderry, NH (US)

Correspondence Address: LAHIVE & COCKFIELD, 28 STATE STREET, BOSTON, MA, 02109, US

(73) Assignee: Atrium Medical Corporation, Hudson, NH

(21) Appl. No.: 10/187,074

(22) Filed: Jun. 28, 2002

Related U.S. Application Data
(60) Provisional application No. 60/355,557, filed on Feb. 8, 2002.

(51) Int. Cl. A61F 2/06

(52) U.S. Cl. 604/891.1; 623/1.15; 623/1.42

(57) ABSTRACT

An implantable medical device having a removable polymeric drug delivery panel electrostatically coupled to a surface of a radially expandable structure is provided. The removable polymeric drug delivery panel provides a microporous structure suitable for embedding one or more bioactive agents to allow for kinetic release of the agent or agents at a desired location within a hollow fluid body organ. The removable polymeric drug delivery panel is characterized as having a relatively large and flat surface area to allow for extended or high volumes of kinetic release potential at the site.

### RELATED APPLICATIONS

This application claims the benefit of Provisional Application Serial No. 60/355,557; filed Feb. 8, 2002.

### TECHNICAL FIELD OF THE INVENTION

The present invention generally relates to implantable medical devices, and more particularly, to implantable medical devices for delivering a bioactive agent.

### BACKGROUND OF THE INVENTION

Restenosis, is the reclosure of a previously stenosed and subsequently dilated peripheral or coronary vessel. Restenosis continues to be a problem with non-medicated medicated mechanically deployable intraluminal support structures, such as balloon expandable and self-expandable stents, and other surgically implanted medical devices, such as vascular grafts, tracheal/bronchial implants, prostate and urethral implants, and synthetic soft tissue implants. A common cause for restenosis is mechanical stress induced cell injury due to the intraluminal support structures. Consequently, a naturally occurring chemo-receptor response occurs, which ultimately activates the process of smooth muscle cell proliferation in and around an effected zone of mechanical tissue injury to cause restenosis.

Recently, published animal research and recent human clinical trials using a combination of drug coated and drug eluting implantable medical devices, have demonstrated significant improvements in restenosis in both animals and humans. The drugs selected in these studies include immunosuppressive and chemotherapeutic drugs, such as Paclitaxel, Sirolimus, Tacrolimus, Everolimus, Taxane, and Rapamycin. These published results demonstrate the ability to reduce, and possibly eliminate the occurrence of smooth muscle cell proliferation, cell replication and restenosis following mechanical injury to endothelialized body fluid organ tissue with drug eluting stents.

One such drug eluting device, is a balloon expandable stent that utilizes multiple drug impregnated elastomeric polymer bands or sleeves that are bonded to the outer surface of a cylindrical tube or stent. The elastomeric band or sleeve are radially spaced about the outer surface of the stent. The elastomeric band stretches radially around the stent as the balloon expandable cylinder stent is expanded by inflation of a percutaneous transluminal coronary angioplasty (PTCA) balloon catheter to a larger fixed diameter inside a blood carrying vessel. Once inflated, the plastically deformable struts in the cylinder wall hold the stent at the second fixed large diameter, which in turn holds the drug impregnated elastomeric bands in a second larger fixed diameter while remaining bonded to the surface of the cylindrical stent. The drug impregnated polymer bands allow the medication to leach out from the elastomeric material in a fixed and radial condition after stent deployment within the vessel.

Studies with the bonded elastomeric polymer band devices indicate that such devices provide an initial “blast” of medication, after which the amount of medication provided quickly declines over time. Small animal histological studies with this drug eluting elastomeric polymer band drug delivery stent have shown cellular levels of released medication, that are detectable for up to one week in and around the drug containing elastomeric polymer band. It has also been shown that when such devices have been implanted in humans, such drug eluting devices have shown sufficient clinical suppression of smooth muscle cell proliferation or restenosis for up to six months by the early release activity of the drug which was eluted from the individual radially fixed elastomeric polymer bands.

Another such drug eluting device utilizes a drug eluting polymer coating placed directly onto and around the entire surface of a cylindrically shaped slotted stent. The drug is impregnated and/or coated directly onto the entire surface of the cylindrical stent like paint or other coating material. The polymer coating contains a bonding agent for permanent adherence to the stent surface and an immunosuppressive or bioactive drug, such as Sirolimus (Rapamycin or Rapamune) or with an antineoplastic medication, such as Pacilataxel or Tacrolimus. The polymer coating bonding agent adheres the drug to the cylindrical surface of the stent. A method suitable for coating an implantable device in this manner is described in U.S. Pat. No. 6,153,252 of Hossainy et al. Such devices have shown equally promising results with a thin coating of a bonded drug polymer complex whose active drug ingredient has been immobilized within a bonding agent adhering the drug to the cylinder surface of the stent. An example of such a coatable implantable device is described in U.S. Pat. No. 6,273,913 of Wright et al.

Another application or method of coating an implantable device with an immunosuppressive or bioactive drug is dipping the cylindrical stent into a bonding agent containing a therapeutic agent prior to crimping the stent to a delivery device, such as a balloon catheter. The dipping technique can be made to preferentially coat one side or surface of the stent, or both sides or surfaces of the stent like a coated chain link fence. However, the painted area or bonded area is limited to the available surface area of the stent, and thus, the drug release is limited due to the limited surface area of the porous metal cylinder stent struts and thin nature of the coating. Thick coatings tend to crack and delaminate upon flexing and or strut movement during coated stent deployment.

In brief, there exists, at least four known types and methods of making a drug eluding stent by coating a cylindrical stent with immunosuppressive or bioactive medications. The current drug eluting stents provide a surface effect release mode from one or more bonded materials or coatings. Such drug eluting surface effects are accomplished by immobilizing the active drug ingredient into a bio-erodable or absorbable polymer coating or bonding agent which is made part of the stent through fusing means, grafting means impregnation means, or a combination thereof to bond the immobilized medication directly to the metal surface of the stent. Other drug eluting surface effects are accomplished by elastomeric polymer sheath attachment directly into, and around the porous metal struts, or by polymer tubular sleeve band attachment surrounding the outer radial cylindrical surface of the stent.

Yet another common method to deploy drugs within the lumen of a vessel is described by U.S. Pat. No. 5,342,348 of Kaplan and U.S. Pat. Nos. 5,725,567, 5,871,535, 6,004,346, 5,997,468 of Wolf et al. These patents describe the use of stent with at least one flexible and round spherically shaped polymeric filament attached within and made part of the support elements of the stent. The round spherically shaped polymeric filaments described by the wolf patented device are compounded with a drug so that the drug is delivered to the vessel lumen by the stent structure upon deployment. The use of a drug containing thread, monofilament or braided fiber is described as being attached and made part of the helical oriented filament stent construction. Use of such monofilaments or filaments in general do not possess a suitable amount of drug eluting capability because the filaments have a round or spherical and thread like shape, and the reduction in effective surface area caused by the interweaving of the round filaments with the structural elements of the stent. As such, only a limited portion of the surface area of the helically oriented round filaments come into direct contact with the inner wall surface of the vessel or organ.

The now available surface release drug delivery methodologies have shown some feasibility of reducing restenosis or smooth muscle cell hyperplasia via balloon expanding stents or self-expandable stent technology having bonded drug eluting surfaces or drug eluting filaments. It is the objective of this invention to improve the duration, delivery, dosemetric control, of the implantable medical device in contact with the lesion to deliver the bioactive agent. It is a further objective of this invention to improve the performance of drug delivery about a radially expandable stent without substantially effecting the surface profile and flexibility of a stent when deployed within mechanically injured tissue or mechanically supported organ, via improved kinetic drug release with a non-bonded, three-dimensional drug delivery device described as a longitudinal oriented medicated polytetrafluorethylene (PTFE) panel.

Since filament drug delivery elements, polymer bonding and dip coatings, and or along with spray or plasma deposited coatings are subject to the available surface area of a structural stent element for attachment means, such methods are restricted to the amount of bonding agent and the amount of medication that can be loaded onto the surface the structural elements of a porous cylindrical stent. In other words, if one can only provide a drug delivery polymer to the effective surface area of a stent structure that can be bonded or coated, the kinetic drug delivery potential can only be equal to the amount of surface area that can be bonded, times the coating's thickness. If one were to provide a thin layer to the entire surface of the structural elements of a slotted or coiled metal strut stent, then the kinetic drug release potential could only be controlled by the amount of surface area of the available polymer bonded as the medication delivery means. If a thick layer of agent is applied, then of course the amount of kinetic drug release potential could potentially be increased by that thickness factor. But, thicker coatings cannot effectively go beyond some plastic limit as defined, because such metal strut devices are deformed and expanded from a compacted first diameter and then to a second larger plastically deformed and fixed larger diameter. The drug eluting polymer coatings cannot increase their surface area or increase their kinetic drug release potential other than by making the coating thicker.

Such coated devices are considered to be drug immobilizing stents, as only some of the drug is available for tissue contact surface release, or surface activation. Most immobilized bonding agents tie up or hold back medication with only small quantities of drug being released principally from one surface dimension. Therefore, most coated and filament attached drug eluting cylindrical stents exhibit only small amounts of actual drug release on the surface of the stent. Such drug release volumes can be increased by making the bonded filament larger or by providing thicker coating, but such increases in coating or filament thickness potentially reduce the filaments and the coating's flexibility and adhesion, further reducing the stent's flexibility, trackability and ability to expand uniformly during deployment without cracking or delaminating. Consequently, the cylindrical stent's ability to pass through and reach the targeted narrowed lesion designation is drastically reduced with thicker coatings or with helically oriented external threads or round filaments or both.

Other drug delivery devices, such as those that incorporate one or more cylindrical polymer bands are constructed of a bonded elastomer matrix that has an active drug ingredient such as Taxane, immobilized into its bonding agent (e.g. polyurethane). Immunosuppressive drugs used in this format have shown promising early clinical results, even though such drugs are limited by the amount of elastomer surface area by which the polymer band is bonded directly to the porous metal cylinder stent prior to balloon expansion and deployment inside a vessel. After balloon expansion of the stent having the drug containing elastomer bands, the elastomeric bands remain permanently bonded to the metal surface of the stent.

Such elastomeric cylindrical bands or material rings or circumferential coverings must be permanently bonded directly to the stent surface to allow the elastomer material to stretch uniformly during stent deployment. Without bonding the elastomer bands or material rings to the surface of the stent, they would move away from their intended fixed position during stent expansion and permanent strut deformation. In order to increase the medication surface activation for kinetic release, the effective elastomeric bonded surface area must be increased, or the thickness of the elastomeric banding zone must be increased or both. Therefore, the cylindrical elastomeric banded drug delivery device is dependent upon the available surface area of elastomeric material bonded directly onto the surface of the cylindrical stent, to modulate or control, or both, the amount of kinetic drug release potential.

It is also known that radial coating or bonding such tubular PTFE film polymers directly onto porous metal cylinder stent structures can cause the stents to become stiff, especially in their compacted first diameter for intraluminal delivery, which, in turn, potentially adversely effects the stent's ability to track without guide catheter resistance, or effects the stents flexibility. In addition, the radial coating or bonding also effects the stent's ability for a uniform strut expansion and deformation following deployment. The mere process of bonding even soft and flexible polymers, including thin PTFE tubular film materials around an entire articulating strut segment in a radial fashion, can significantly reduce a porous metal stent's flexibility and plastic deformation for uniform stent placement within a lumen of a blood vessel. Uniform stent expansion is required so as to not disrupt laminar blood or fluid flow through the expanded or deployed stent device. In other words, the more surface area that a drug eluting filament or polymer coating is permanently attached or bonded to, the less flexible and functional the articulating struts of the cylinder stent structure become, which, in turn, causes stent foreshortening or luminal diameter recoil after intraluminal deployment, placement or both.

Since many of the current drug eluting devices are limited in their kinetic drug release potential due to the amount of polymer coating available on the surface of the metal strut surface, these drug eluting devices provide medication out from one planar exposed surface. As such, the drug release potential can, at best, be increased by covering more open surface area of the porous metal stent or by making the filament or bonded coating thicker, at the sacrifice of stent flexibility and trackability.

The drug eluting stents that have shown initial favorable clinical results are limited in their ability to provide extended or higher volumes of kinetic drug release potential, due to their dependence on the amount of filament surface or coating surface or bonding surface area that can come into direct contact with the inner wall surface of the tissue and along the longitudinal planar surface of the porous metal cylindrical stent. In order for either of these aforementioned methods to provide additional kinetic drug release activity, such filament, coating and bonding agents can only be increased in material mass or thickness to increase their effective drug release potential. For example, a drug eluting sleeve that provides a near total covering over the entire porous metal cylindrical stent is described by U.S. Pat. No. 5,383,928 of Scott et al. Such options are often not always practical as thicker encompassing sheath coverings, or multi-filament sheath covers and or continuously radial coatings, which are attached and bonded directly to the individual surfaces of the articulating metal struts, significantly reduce the overall flexibility of the metal strut stent and subject the stent to the risk of delamination and separation of the covering, filament or bonded drug coating material due to the increased rigidity of the cylindrical stent during flexing and manipulation that occurs during stent deployment. Furthermore, if the thickness drug coating or sheath increases too much, the stent would be at risk of being under sized which can cause significant flow turbulence upon insertion within a lumen. Therefore, increasing a drug immobilizing bonding agent's material mass, thread and filament diameter or thickness would have a dramatic negative effect on the stent's performance and ability to track along and fit into and glide through the narrow passageway of a stenotic tubular organ lesion, and further perform its intended purpose of creating a non-restricted flow passageway. Thicker drug coatings, threads and filaments, sheaths or radial placed individual bands also limit the stent's ability to uniformly expand to a desired fixed larger diameter due to increased stent wall thickness by the encompassing thread, filament, polymer cover, sleeve or elastomeric band. Reduced trackability, or the ability of the stent to pass-along-and-thru a narrow lesion, can also be significantly reduced and hindered by use of a thick or stiff drug eluting thread, filament, or coating, or bonded drug polymer sleeve about the stent. Moreover, current drug eluting polymer coated stents fail to provide a means for cells to grow into the drug delivery polymer material to further stabilize the biocompatible drug delivery polymer device, following drug release.

Hence, it is our objective to provide a novel drug delivery method and device to allow cells to grow into a delivery mechanism to stabilize the device at a deployed location and to increase the kinetic drug release potential at a desired location within the body where an expandable stent or stent graft is placed without the need for chemical bonding or dependence on the amount of surface area of a cylindrical structure, and further without substantially reducing the flexibility, or increasing the stent's overall wall surface profile, or adversely effecting the stent's trackability, or ability to uniformly expand during expansion or deployment of the implantable medical device.

This drug delivery device and method of manufacture can be suitable for a wide spectrum of bioactive agents or medications, and can be made suitable for many different stent strut geometrics which may require a greater kinetic release potential than those employed by current filament attached stents, coated stents and drug eluting polymer techniques; including bonded radial elastomer sleeves, individual drug eluting polymer rings, bands, threads or filaments that can be made part of a drug eluting tubular construction, and to make the device microporous for cells to grow into the PTFE panel following drug release to help stabilize the PTFE panel into the cellular wall surface of the organ tissue.

Therefore, the below described drug delivery device and method of manufacture provide a novel technique to increase kinetic drug release potential without substantially sacrificing the overall flexibility and trackability of the articulating metal strut members of an expanding implantable device. It is an objective of this device to provide prolonged and controlled drug release without the use of a permanently attached or woven, knitted or braided in filament elements, permanent or bioerodable coatings or bonding agents or polymer blends that are fastened to or made permanently part of the structural surface or construction of an implantable stent device, such as a structural stent strut element.

It is another objective of this drug delivery device, to not effect the uniform expansion or plastic deformation of an implantable device, for example, a radially expanding stent structure, and to provide enhanced kinetic drug release potential in and around the deployment area of the stent after fixation to tissue within a lumen of a hollow fluid carrying organ. The enhanced kinetic drug release potential provided by a drug-containing and non-bonded medicated PTFE panel is also temporarily and electrostatically coupled to a surface of the stent.

It is another objective of this invention to provide a lower coefficient of friction to a portion of the outer surface of a crimped metal stent with a medicated PTFE panel without effect to the uniform expansion or plastic deformation of the stent structure. It is a further objective to provide a drug-containing and non-bonded medicated PTFE film panel for enhanced kinetic drug release potential in and around the deployment area of the stent after fixation to tissue.

It is another objective of the present invention to provide a medicated PTFE panel for electrostatic coupling to an expandable device that can be effectively incorporated by and penetrated by tissue and cells during and after kinetic drug release from all surfaces of the panel.

It is another objective of the present invention to provide over a full stent length or over a partial stent length a drug delivery mechanism via one or more longitudinal strips of medicated and panelized PTFE material capable of prolonged kinetic drug release, without coating, strut weaving, bonding, or radial elastomeric banding directly to the entire radial surface of a porous metal cylindrical stent.

It is another objective of the below described invention to apply a medicated PTFE panel to a stent after the tubular stent has been fully crimped down, or compacted, and installed into or onto a catheter delivery mechanism, or placed into a delivery catheter such as used with PTCA and with peripheral transluminal angioplasty (PTA) folded balloon catheters and with self expanding stent catheter delivery platforms.

It is a further objective of this drug delivery device to advantageously use the inherent electro-negative surface charge properties of the PTFE panel as one means for temporarily coupling at least a portion of the panel to a portion of a fully crimped expandable stent, together with one or more mechanical containment means to the balloon catheter by the stent or stent strut element.

It is another objective of this drug delivery device to use the inherent electro-negative surface charge properties of the medicatable PTFE panel as one means for temporary attachment to a portion of both a folded polymeric balloon and a fully crimped cylindrical stent, together with one or more temporary mechanical pinching means to the balloon catheter by the stent.

It is another objective of the below described drug delivery device to provide two or more bioactive agents, with different pharmaceutical effects, and independent drug eluting rates of delivery-of medication relative to each other, by application of two or more independent medicatable PTFE panels applied to a portion of an implantable expandable device such as a stent.

It is another objective of the below described drug delivery device to allow the physician to tailor or customize the dosemetric amount of a selected bioactive agent through selection of a length or quantity of medicatable panels applied to an implantable and expandable medical device. Thus, giving the physician the ability to customize dosage of a selected bioactive agent based on circumstances such as treatment protocol, patient's condition and the like. The dosage amount is customized by addition or removal of one or more removable dosemetric controllable medicated panels or by selecting a desired length of the panel. The medicated panel provides a means for enhanced dosemetric drug control not currently possible with known drug delivery stent products.

It is another objective of this invention to provide cellular in growth into the drug delivery panel following drug release out from the three dimensional drug eluting material without effect to the stent struts.

### SUMMARY OF THE INVENTION

The present invention provides an implantable medical device having a removable polymeric drug delivery panel electrostatically coupled in a temporary manner to at least a portion of a radially expandable structure. The removable polymeric drug delivery panel is characterized by a seamless construction of fluoropolymer material, such as expanded polytetrafluoroethylene (ePTFE), preferably constructed in a closed three dimensional geometric form bounded by substantially straight surfaces.

The use of the fluoropolymer material for the removable polymeric drug delivery panel provides an implantable medical device having a biocompatible construction that is suitable for numerous uses including a drug delivery vehicle for the treatment of body vessels, organs and implanted grafts. The orientation of the removable polymeric drug delivery panel along a central longitudinal axis of the radially expandable structure provides extended or high volume of kinetic drug release potential due to the microporous structure of the drug delivery panel and its significant surface area contacting a lumen of a hollow body organ. The electrostatic coupling of the removable polymeric drug delivery panel to the radially expandable structure advantageously avoids the need for a polymer or other bonding agent while allowing the implantable medical device to uniformly expand to a desired fixed large diameter. Moreover, the electrostatic coupling allows the implantable medical device to maintain trackability or the ability of the implantable medical device to pass along and through a narrow lesion without being significantly hindered by a stiffening of the implantable medical device due to a compounded filament, polymer coating or one or more bonded polymer sleeves radially spaced about the device.

According to one aspect of the present invention, the removable polymeric drug delivery panel extends along the central longitudinal axis of the radially expandable structure from a first end portion to a second end portion. The dimensioning of the removable polymeric drug delivery panel so that it extends along the central longitudinal axis ensures that following deployment of the radially expandable structure within a fluid carrying organ or space a substantial portion of the outer surface of the structure within the fluid containing organ or space is free of the drug delivery panel. The removable polymeric drug delivery panel is also characterized as having one or more contourable surfaces, that is, at least a first surface of the removable polymeric drug delivery panel is contourable to a radial dimension of the radially expandable structure to which the removable polymeric drug delivery is applied. Specifically, the removable polymeric drug delivery panel includes a first contourable surface having an electrostatic charge potential, the first surface is adaptive to a portion of a curvature of the outer surface of the radially expandable structure. The drug delivery panel also includes a second contourable surface. The second surface is adaptive to a portion of a curvature of an inner lumen surface within the fluid containing organ or space upon deployment of the radially expandable structure therein.

In accordance with a further aspect of the present invention, a method is provided for manufacturing an expandable implantable medical device constructed with a removable polymeric drug delivery element of a fluoropolymer material such as, for example, ePTFE. The method includes the step of providing a radially expandable element having a central longitudinal axis and an outer surface. The removable polymeric drug delivery element is electrostatically coupled to a portion of the outer surface of the radially expandable element along the central longitudinal axis. The removable polymeric drug delivery element extends along the central longitudinal axis from a first end portion to a second end portion of the radially expandable element to cover a portion of the element's outer surface. The result is an expandable implantable medical device that is radially expandable from a first reduced diameter to a second larger diameter upon application of a radially deployment force from a deployment mechanism without the electrostatically coupled removable polymeric drug delivery element substantially hindering uniform deployment.

To ensure proper placement of the removable polymeric drug delivery panel within a hollow fluid containing organ or space the method optionally provides the step of attaching a fastener element to a portion of the radially expandable element. This allows the removable polymeric drug delivery element to be temporarily and mechanically fastened to a portion of the wall of the radially expandable element to reduce the risk of movement during insertion of the implantable medical device through a lesion or during transport within the hollow fluid carrying organ or space. The fastener element can also be adapted to mechanically fasten a portion of the drug delivery element to the wall surface of the radially expandable element and to a portion of the balloon deployment delivery catheter. Further, the method provides the step of loading the removable polymeric drug delivery element with a therapeutic amount of a selected bioactive agent for local administration of the bioactive agent at a selected treatment site within a fluid containing organ space, lumen or opening.

The loading, compounding or infusing of the removable polymeric drug delivery element with a selected bioactive agent provides for extended or high volume of kinetic therapeutic release potential without significantly impacting mobility, flexibility, deployability, expandability or the like of the radially expandable element. Furthermore, the loading of the removable polymeric drug delivery element with a therapeutic amount of a selected bioactive agent provides dosemetric control for the selected agent. For example, factoring the absorbability of the removable polymeric drug delivery element for the selected bioactive agent, the length dimension and, optionally, the width dimension of the removable polymeric drug delivery element can be sized to provide a desired dosage of the agent. As such, a physician is able to size the polymeric drug delivery element to suit on the selected bioactive agent, the treatment being performed and other factors that require the physician to prescribe a particular dosage amount, such as the patient's age, weight and overall health.

Moreover, because the polymeric drug delivery element is removable and electrostatically coupled to the radially expandable element, a physician can select, size and load the polymeric drug delivery element with a selected bioactive agent immediately before the medical device is implanted in the patient. As a consequence, hospitals and other medical treatment facilities are not burdened with inventory costs associated with stocking a pharmacy with an abundance of implantable medical devices having coated thereto various bioactive agents with various varying thickness so as to provide a physician with a device having the prescribed dosage. As such, the treating physician can select a desired stent and attach thereto a removable polymeric drug delivery element of a length suited for the dosage prescribed so as to customize the treatment regimen for the patient. The selecting of the desired length of the removable polymeric drug delivery element in combination with the selecting of a particular bioactive agent provide the administering physician with a novel dosemetric control mechanism for the bioactive agent.

In accordance with another aspect of the present invention, a stent for bioactive drug delivery within a hollow organ tissue is provided. The stent is characterized as an expandable tubular element having an inner passage, a longitudinal axis and an outer wall. The stent also includes at least one removable and longitudinally oriented microporous polymeric panel element electrostatically coupled to at least a portion of the outer wall of the tubular element along its longitudinal axis. The polymeric panel is characterized by a substantially thin and flat and seamless construction of fluoropolymer material, such as ePTFE. The polymeric panel element extends parallel to and along the longitudinal axis from a distal portion to a proximal portion of the expandable tubular element. This allows the expandable tubular element to include a longitudinally porous surface contact portion capable of providing bioactive drug delivery to an inner surface of a hollow fluid carrying organ. Moreover, the microporous polymer panel includes a first and second surface profile with each surface profile capable of adapting to a curvature that substantially matches a surface profile of the outer wall of the expandable tubular element before and after deployment within the hollow organ targeted for therapeutic treatment. In addition, the microporous structure of the polymeric panel element allows for cellular growth into the polymer panel following elution of the bioactive agent. Consequently, the microporous polymeric panel element also serves as a stable platform about which cellular regeneration can take place.

The removable microporous polymeric panel element is also characterized as having a bioactive agent compounded therein and upon expansion of the expandable tubular element at least one surface of the panel element maintains substantially continuous direct contact with an inner wall surface of a selected hollow organ. Exemplary treatment applications of the present invention application includes dilation of stenoic blood vessels in a percutaneous transluminal angioplasty procedure (PTA), removal of thrombi and emboli from an obstructed blood vessel, urethra dilation to treat prostactic enlargement due to benign prostatic hyperplasia (BPH) or prostatic cancer, and generally restoring patency to body passages such as blood vessels, the urinary tract, the intestinal tract, the kidney ducts or other body passages.

In a further aspect of the present invention, a medical device for administering a bioactive substance to a location within a fluid containing organ is provided. The medical device is characterized as having a microporous bioactive substance delivery panel attachable to a surface of a structure suitable for delivering the microporous panel to a location within the fluid carrying organ. The microporous panel is adapted to include a number of surfaces and the panel having a height dimension significantly less than a length dimension and a width dimension. At least one of the surfaces of the microporous delivery panel includes an electronegative charge sufficient for temporary attachment to a surface of the delivery structure. The height dimension, the width dimension and the length dimension of the microporous delivery panel are characterized as a dosage indicator or dosage control feature for indicating or controlling the dose of the bioactive substance compounded therein. The microporous panel is characterized as a fluoropolymer panel, such as, ePTFE. The microporous bioactive delivery panel is further characterized as contourable to a surface topology and curvature of the delivery structure to which it is attachable. The panel is contourable and attachable to the delivery structure without impeding operation of the delivery structure during deployment within the fluid carrying organ.

### BRIEF DESCRIPTION OF THE DRAWINGS

These and other features and advantages of the present invention will be more fully understood by reference to the following detailed description in conjunction with the attached drawings in which like reference numerals refer to like elements through the different views. The drawings illustrate principals of the invention and, although not to scale, show relative dimensions.

FIG. 1 is a perspective view of an implantable medical device according to the teachings of the present invention.

FIG. 1A is a cross section of a removable polymeric drug delivery panel according to the teachings of the present invention.

FIG. 2A is an end view of the implantable medical device of FIG. 1.

FIG. 2B is a side elevational view of a cross section of the implantable medical device of FIG. 1.

FIG. 3 is a side elevational view of a stent device suitable for use in the present invention.

FIG. 4 is a side elevational view of a stent having multiple removable polymeric drug delivery panels according to the teachings of the present invention.

FIG. 5 is a side elevational view of a balloon catheter coupled to a stent having a removable polymeric drug delivery panel according to the teachings of the present invention.

FIG. 6 is a side elevational view of an illuminable stent graft having a removable polymeric drug delivery panel according to the teachings of the present invention.

FIG. 7 is a side elevational view of a catheter having a removable polymeric drug delivery panel according to the teachings of the present invention.

FIG. 8 is a perspective view of a graft having a removable polymeric drug delivery panel according to the teachings of the present invention.

FIG. 9 is a perspective view of a removable polymeric drug delivery panel according to the teachings of the present invention.

FIG. 10 is a perspective view of a removable polymeric drug delivery panel according to the teachings of the present invention.

FIG. 11 is a perspective view of a removable polymeric drug delivery panel according to the teachings of the present invention.

FIG. 12 is an end view of the removable polymeric drug delivery panel according to the teachings of the present invention.

FIG. 13 is a top view of a removable polymeric drug delivery panel according to teachings of the present invention.

FIG. 14 is a top view of a removable polymeric drug delivery panel according to teachings of the present invention.

FIG. 15 is a side elevational view of a cross section of a removable polymeric drug delivery panel having a varying thickness according to the teachings of the present invention.

FIG. 16 is a flow chart illustrating the steps of manufacturing an expandable implantable medical device according to the teachings of the present invention.

FIG. 17 is a flow chart illustrating the steps of infusing a removable polymeric drug delivery panel with a bioactive agent according to the teachings of the present invention.

### DETAILED DESCRIPTION

The illustrative embodiment of the present invention provides an implantable medical device having a removable polymeric drug delivery panel electrostatically coupled in a temporary manner to an outer surface of a radially expandable structure to release a bioactive substance at a desired location within a hollow fluid carrying body organ. In the illustrative embodiment, the implantable medical device includes at least one removable polymeric drug delivery panel formed of a fluoropolymer material, such as expanded polytetrafluoroethylene (ePTFE). The microporous structure of the ePTFE is able to hold and deliver an appropriate amount of the bioactive agent to provide the ability to extend or deliver higher volumes of kinetic drug release potential at a selected treatment site to prevent restenosis or intima hyperplasia.

Before continuing with the detailed description of the illustrative embodiment, it is first helpful to define a few terms.

As used herein, the term “implantable medical device” means any material or device that is capable of being inserted in a fluid carrying organ, and includes catheters, stents, grafts and other like devices or instruments.

As used herein, the term “bioactive agent” refers to any substance capable of producing an effect, whether physical, chemical or bioactive in a human or animal. Table I listed below provides an exemplary list of bioactive agents suitable for use in an illustrative embodiment of the present invention. Table I is not meant to limit an illustrative embodiment of the present invention to one or more of the exemplary bioactive agents listed, but rather is meant to illustrate the ability of the illustrative embodiment to support a variety of treatment protocols for a variety of therapeutic applications within a fluid carrying organ.

| Class | Examples |
|---|---|
| Antioxidants | Lazaroid, Probucol, Vitamin E |
| Antihypertensive Agents | Diltiazem, Nifedipine, Verapamil |
| Antiinflammatory Agents | Glucocorticoids, Cyclosporine, NSAIDS |
| Growth Factor Antagonists | Angiopeptin, trapidil, suramin |
| Antiplatelet Agents | Aspirin, Dipyridamole, Ticlopidine, |
| | Clopidogrel, GP IIb/IIIa inhibitors, |
| | Abcximab |
| Anticoagulant Agents | Heparin (low molecular weight and |
| | unfractionated), Wafarin, Hirudin |
| Thrombolytic Agents | Alteplase, Reteplase, Streptase, Urokinase, |
| | TPA |
| Drugs to Alter Lipid | Fluvastatin, Colestipol, Lovastatin |
| Metabolism (e.g. statins) | |
| ACE Inhibitors | Elanapril, Fosinopril, Cilazapril |
| Antihypertensive Agents | Prazosin, Doxazosin |
| Antiproliferatives and | Cochicine, mitomycin C, Rapamycin, taxols, |
| Antineoplastics | Everolimus, Tacrolimus, Sirolimus |
| Tissue growth stimulants | Bone morphogeneic protein, fibroblast |
| | growth factor |
| Gasses | Nitric oxide, Super Oxygenated O₂ |
| Promotion of hollow organ | Alcohol, Surgical Sealant Polymers, |
| occlusion or thrombosis | Polyvinyl particulates, 2-Octyl |
| | Cyanoacrylate, Hydrogels, Collagen |
| Functional Protein/Factor | Insulin, Human Growth Hormone, Estrogen, |
| Delivery | Nitric Oxide |

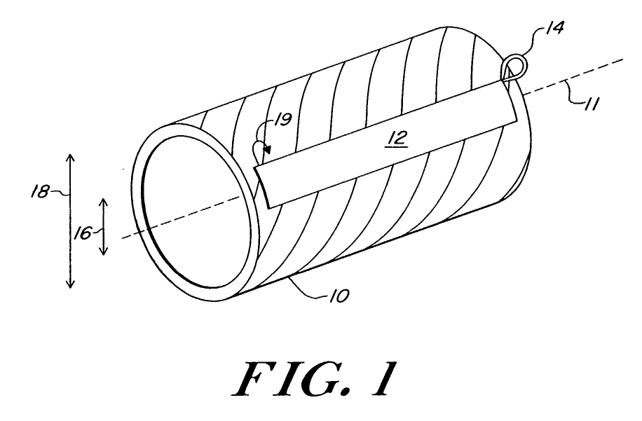

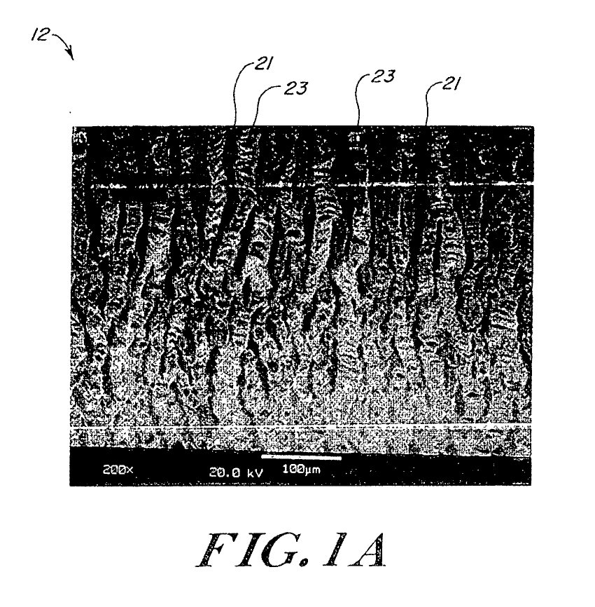

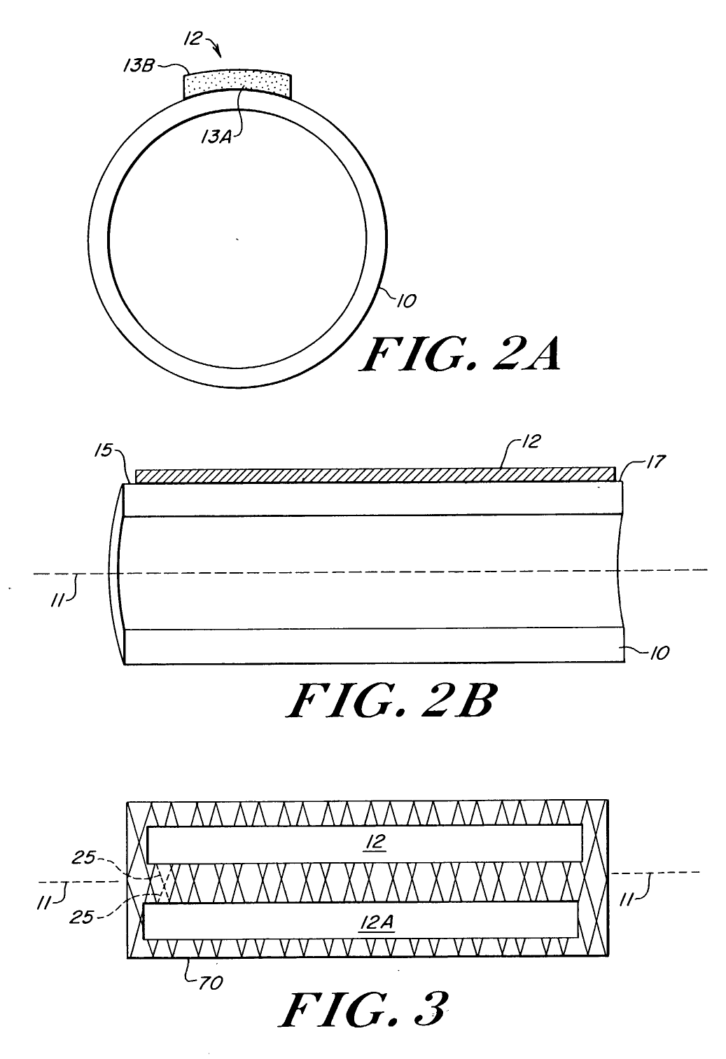

With reference to FIGS. 1, 2A and 2B, the implantable medical device of the illustrative embodiment includes a radially expandable structure 10 having electrostatically coupled thereto a removable polymeric drug delivery panel 12 constructed of a microporous material such as expanded fluoropolymer material. The implantable medical device provided by the present invention is suitable for a wide range of in-vivo applications including, for example, therapeutic treatment of body passages such as blood vessels, the urinary tract, the intestinal tract, the kidneys, ducts, and other passages with one or more selected bioactive agents. Specific therapeutic treatment examples include the delivery of a bioactive agent to a selected site within a blood vessel to significantly reduce, or potentially eliminate, restenosis. The implantable medical device of the illustrative embodiment advantageously allows a treating physician to infuse the removable polymeric drug delivery panel 12 with a selected bioactive agent for the administering of the bioactive agent to a site within a fluid containing organ without impacting the uniform expansion of the implantable device at the treatment site.

The radially expandable structure 10 is deployable within a hollow fluid carrying organ upon application of an expansion force to expand the structure from a first, reduced diameter 16, to a second increased diameter 18. The radially expandable structure 10 generally exhibits no elastic properties, that is, it retains its shape following expansion. Optionally, the radially expandable structure 10 is adaptable to have elastic properties, that is, the radially expandable structure 10 is held in a contracted position for placement within a hollow fluid carrying organ. As such, once the radially expandable structure 10 having elastic properties is placed at the desired location, the tension holding the structure in its contracted position is released to allow the radially expandable structure 10 to expand and cause the removable polymeric drug delivery panel 12 to contact an inner lumen wall of the hollow fluid carrying organ. Nevertheless, those skilled in the art will recognize that the removable polymeric drug delivery panel 12 is suitable for use with an implantable medical device that does not have a radially expandable structure, for example, a graft or other like implantable medical device that are discussed below in more detail.

The radially expandable structure 10 can be composed of a variety of biocompatible materials. Such materials include, but are not limited to, stainless steel, silver, tantalum, gold, titanium, tungsten, platinum, and polymers, such as polyether sulfone, polyamide, polycarbonate, polypropylene, high molecular weight polyethylene, carbon fiber and the like. In addition, the radially expandable structure 10 is adapted to include an open or perforated structure, such as a helically wound or serpentine wire structure. The turns or curves in the wire forming the perforations in the radially expandable structure 10.

The removable polymeric drug delivery panel 12 is a singular, unitary article of generally homogeneous material of uniform shape. The removable polymeric drug delivery panel 12 is characterized by a seamless construction of an elastic expanded fluoropolymer material having substantially flat top and bottom surfaces along with substantially flat side and end surfaces. The removable polymeric delivery panel 12 provides a microporous structure suitable for the delivery of bioactive agents in a predictable manner. The panel shape of the removable polymeric drug delivery panel 12 provides a distinct profile that allows a physician to maximize the amount of bioactive agent delivered to a selected area within a hollow fluid carrying organ. Moreover, the removable polymeric drug delivery panel 12 and the manner in which it is coupled to the radially expandable structure 10 avoids significant reduction in the overall flexibility of the radially expandable structure 10, which, in turn, also avoids other significant risks associated with an implantable medical device lacking sufficient flexibility, for example, delamination and separation of a radical covering, a filament, or a coated or bonded material. Furthermore, the electrostatic manner in which the removable polymeric drug delivery panel 12 is coupled to a portion of the radially expandable structure 10 allows for dosemetric control of a selected bioactive agent by a physician through the step of trimming or selecting an appropriately sized removable polymeric drug delivery panel 12 for the treatment protocol.

FIG. 1A illustrates a cross section of the removable polymeric drug delivery panel 12. The cross sectional view illustrates a porous structure of the removable polymeric drug delivery panel 12 that is suitable for implantation in the human body. The porous structure of the removable polymeric drug delivery panel 12 consists of a microstructure having a generally fragmented appearance in which larger relatively solid “nodes” 21 of material are held together by less substantial and more numerous “fibrils” 23 of the material that cross or criss-cross the space between nodes. The removable polymeric drug delivery panel 12 is essentially biologically inert and the fibrils 23 are of such a small diameter, for example, 10 to 150 angstroms, that cellular material of a hollow fluid carrying organ can simply bend the fibers and grow into spaces therebetween. The fibrils 23 can also be sized to prevent cellular in growth, but allow fluid communication two or more cells in the polymeric drug delivery panel 12. Accordingly, the removable polymeric drug delivery panel 12 when having a suitable porous microstructure can serve as an immobilizing platform or anchor about which cellular regeneration can take place. The microporous structure of the removable polymer drug delivery panel 12 not only allows tissue growth into the spaces, but also allows formation of capillary blood vessels and other differentiated tissue.

The removable polymeric drug delivery panel 12 is made porous by fabricating it with a stretching step to develop an internode spacing of between approximately one micron and two hundred microns, preferably about 50 microns, although the precise porosity will depend on factors such as the solubility, viscosity and other properties of the bioactive agent which is to be loaded, compounded, or infused into the removable polymeric drug delivery panel 12. Other factors that effect the porosity of the removable polymeric drug delivery panel 12 include the tissue growth characteristics of the intended treatment site. For example, if the bioactive agent is highly soluble, smaller pores are necessary to control the rate of elution. Similarly, if the tissue at the site of the lesion is highly proliferating and it is decided to inhibit cellular in growth, then pore sizes should also be kept small under several microns, for example less than 10 microns.

While the generic term “porous” and “porosity” have been used, it is understood as used herein, to encompass those measures of porosity customarily used to describe graft and other implantable medical devices of PTFE. Moreover, it is understood that to accurately achieve such small pore sizes the node spacing (distance between adjacent nodes) and the fibril length should each be controlled so that they present the desired porosity. For pore sizes below several micrometers, this generally requires that the node spacing and fibular length each be under about 10 or 20 micrometers.

Continuing to refer to FIG. 1, the removable polymeric drug delivery panel 12 is generally aligned with a central longitudinal axis 11 of the radially expandable structure 10. As illustrated, the removable polymeric drug delivery panel 12 is electrostatically coupled in a temporary manner to a portion of the radially expandable structure 10. In this manner, a first surface of the removable polymeric drug delivery panel 12 contacts at least a portion of the outer surface of the radially expandable structure 10 in a contourable manner; which allows for substantially uniform expansion of the radially expandable structure 10 from the first diameter 16 to the second diameter 18. In addition, the removable polymeric drug delivery panel 12 includes a second contourable surface opposite the first contourable surface that is adaptive to a curvature and topology of an inner wall lumen of a hollow fluid carrying organ following deployment of the radially expandable structure to its enlarged second diameter 18. The thickness of the removable polymeric drug delivery panel 12 is between about 0.1 microns and 150 microns.

The radially expandable structure 10 optionally includes a fastener 14, which is illustrated as a loop fastener, such as a suture or other like thread element suitable for use within a human organ to mechanically secure the removable polymeric drug delivery panel 12 to a portion of the surface of the radially expandable structure 10. The fastener 14 is also configurable as a bendable element made part of the radially expandable structure 10 that bends from a first position to a second position and alternately back to the first position to fasten a portion of the removable polymeric drug delivery panel 12 to the outer surface of the radially expandable structure 10. One skilled in the art will recognize that the radially expandable structure 10 and the removable polymeric drug delivery panel 12 are also capable of being crimped onto a deployment delivery catheter.

The removable polymeric drug delivery panel 12 is well suited for bioactive agent compounding and release of the agent at a desired site within a hollow fluid carrying organ. The removable polymeric drug delivery panel 12 is electrostatically coupled to a portion of the radially expandable structure 10 so as to avoid impeding delivery and deployment of the implantable medical device. With this construction, the removal polymeric drug delivery panel 12 avoids interfere with the operation of the radially expandable structure 10 in either its first diameter 16 or its second diameter 18. As such, the removable polymeric drug delivery panel 12 advantageously allows uniform expansion of the radially expandable structure 10 to a desired fixed larger diameter while maintaining the medicated panel's longitudinal orientation to the stent and allowing the device to pass along and through a narrow lesion without delamination or removal of the medicated panel for delivery of its prescribed therapeutic dosage of a selected bioactive agent. As such, the illustrative embodiment avoids the risk of delamination between the stent and the drug delivery mechanism that is often associated with a utilizing a round filament structure or a drug coating or a bonded polymer sleeve about the stent.

The removable polymeric drug delivery panel 12 has a relatively flat planar surface for interfacing with a portion of the radially expandable strut structure 10 and for contacting the inner lumen wall of a hollow fluid carrying organ as compared to known filament or thread contained stent structures. As such, the structure of the removable polymeric drug delivery panel 12 increases kinetic drug delivery potential due to its three dimensional drug eluting surface area and micro porous surface. Moreover, the relatively, flat planar surface of the removable polymeric drug delivery panel 12 allows the removable polymeric drug delivery panel 12 to act as an immobilizing platform about which cellular regeneration can take place.

The removable polymeric drug delivery panel 12 is essentially biologically inert and is capable of being configured to support cellular regeneration in all of, or a portion of, its microporous structure. For example, the removable polymeric drug delivery panel 12 is configurable so that a selected bioactive agent elutes out of a first portion of the panel's microporous structure and upon elution of the bioactive agent from the first portion of the removable polymeric drug delivery panel 12, cellular regeneration of the hollow fluid carrying organ takes place in the microporous spaces of the first portion of the panel that previously held the selected bioactive agent. In another example, the first portion of the removable polymeric drug delivery panel 12 is configured to elute a selected bioactive agent at a particular rate, but the first portion of the removable polymeric drug delivery panel 12 is configured to prohibit cellular regeneration of the hollow fluid carrying organ into the microporous spaces of the first portion of the panel that held the eluted bioactive agent. The ability to adapt a portion, an entire first surface, or all of the surfaces of the removable polymeric drug delivery panel 12 to support cellular regeneration of a hollow fluid carrying organ upon elution of all or a portion of a selected bioactive agent allows the implantable medical device having coupled thereto the removable polymeric drug delivery panel 12 to promote cellular regeneration at a lesion of a hollow fluid carrying organ if so desired.

Moreover, the ability to adapt or configure all, or portions of the removable polymeric drug delivery panel 12 to allow cellular regeneration in the microporous structure of the panel following elution of all or a portion of a bioactive agent establishes a surface ratio for each surface of the removable polymeric drug delivery panel 12 that expresses a relationship between elution of a selected bioactive agent and cellular regeneration within the microporous structure of a surface of the panel following elution of the selected bioactive agent. The surface ratio is expressed as a percentage and reflects the percentage of surface area for a selected surface of the removable polymeric drug delivery panel 12 is adapted to support cellular regeneration.

In most applications, this surface ratio includes a range from between about 0 percent to about 100 percent with increments in about 5 percent increments. For example, the removable polymeric drug delivery panel 12 can have a first surface ratio of about 100 percent, this indicates that at least a 100% of a first surface of the panel is configured to support cellular regeneration across the first surface upon elution of a selected bioactive agent. In contrast, the removable polymeric drug delivery panel 12 having a first surface ratio of or about 5 percent indicates that the first surface of the panel is configured so that about 5 percent of a first surface area is configured to support cellular regeneration upon elution of all or a portion of a selected bioactive agent. Those skilled in the art will recognize that if about 5% of a surface of the removable polymeric drug delivery panel 12 is adapted to support cellular regeneration, then about 95% of the surface is adapted to not support cellular regeneration.

Those skilled in the art will recognize that the surface ratio discussed above is defined as a percentage of a first surface area of the removable polymeric drug delivery panel 12. Nevertheless, the definition of the surface ratio discussed above can be expanded upon to reflect a percentage of a total surface area of the removable polymeric drug delivery panel 12. For example, a surface ratio of 100 percent would indicate that all of the available surface area of the removable polymeric drug delivery panel 12 is configured to support cellular regeneration upon elution of all or a portion of a selected bioactive agent therefrom in a hollow fluid carrying organ.

FIG. 2A illustrates a first contourable surface 13A of the removable polymeric drug delivery panel 12 contoured to the outer surface of the radially expandable structure 10. FIG. 2A also illustrates that the dimensioning of the removable polymeric drug delivery panel 12 relative to a radial component of the radially expandable structure 10, which advantageously provides a low profile drug delivery mechanism capable of delivering an enhanced kinetic drug release potential without constraining the radially expandable structure 10 during deployment within a hollow fluid carrying organ. As such, the removable polymeric drug delivery panel 12 provides the benefit of allowing the stent to maintain flexibility and transportability within the fluid carrying organ while providing the ability to offer extended or higher volumes of kinetic drug release potential due to its effective surface area in direct contact with the inner luminal wall surface of the hollow fluid carrying organ and due to the microporosity of the removable polymeric drug delivery panel 12. Moreover, the second contourable surface 13B of the removable polymeric drug delivery panel 12 provides a further benefit by maximizing contact area for delivery of the selected agent through the ability to a curative and topology of the inner lumen wall of the hollow fluid carrying organ to result in the patient receiving the maximum therapeutic benefit of the treatment.

By contrast, other implantable medical devices that utilize round filaments or threads or other suture like elements for kinetic drug delivery not only restrict flexibility and expansion of the radially expandable structure due to bulk and interweaving with the strut element of the expandable structure, but also provide a limited surface area for kinetic drug delivery due to their spherical shape and limited surface area for contacting the inner wall of he hollow fluid carrying organ. Moreover, the removable polymeric drug delivery panel 12 is configurable delivery vehicle that can be shaped, sized to match a lesion shape. Furthermore, the ability to configure the removable polymeric drug delivery panel 12 to customize delivery of a bioactive agent allows a medical professional to infuse, load or compound only a portion of the removable polymeric drug delivery panel 12 with a selected bioactive agent. The ability to infuse a portion of the removable polymeric drug delivery panel 12 with a bioactive agent provides an implantable medical device with the capability to deliver more than one bioactive agent to a particular region of a lesion or to deliver a bioactive agent to a particular region of a lesion.

FIG. 2B illustrates that the removable polymeric drug delivery panel 12 advantageously extends along the central longitudinal axis 11 of the radically expandable structure 10 from a first end portion 15 to a second end portion 17. In this manner, the removable polymeric drug delivery panel 12 is able to kinetically deliver one or more bioactive agents held by its microporous structure over a significant longitudinal section of the treatment site. Nevertheless, those skilled in the art will recognize that the length of the removable polymeric drug delivery panel 12 can be reduced based on the amount of bioactive agent thought necessary for the patient's treatment protocol. That is, the removable polymeric drug delivery panel 12 can be sized to a specific patient or a specific treatment protocol by the treating physician, which, in turn, provides healthcare facilities, such as hospitals with a cost significant savings benefit in terms of an inventory reduction, because the facility would no longer have to stock a plethora of coated stents to support the various treatment protocols administered in the facility.

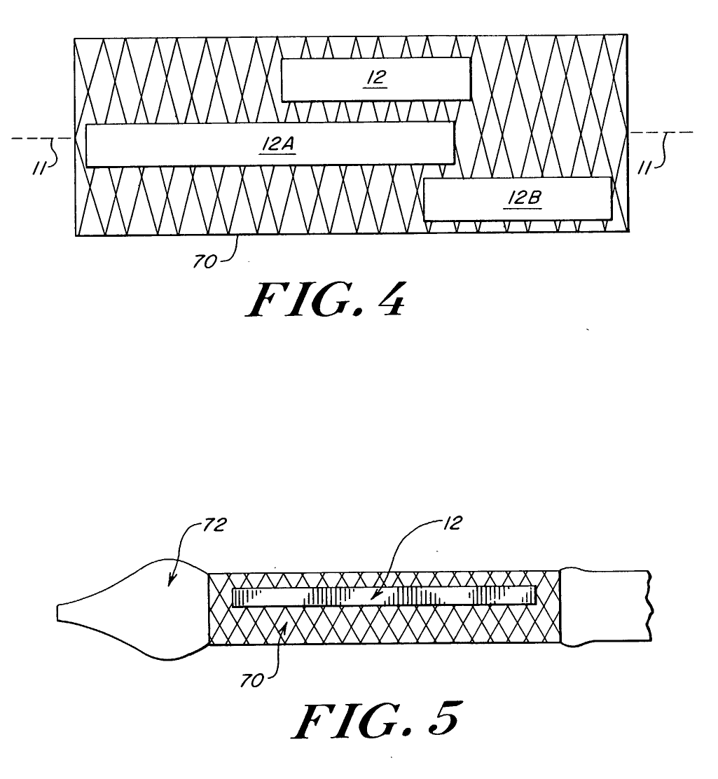

FIG. 3 illustrates a stent 70 having a central longitundal axis 11, a first removable polymeric drug delivery panel 12 and a second removable polymeric drug delivery panel 12A. FIG. 3 illustrates that an implantable medical device, such as stent 70, can be configured to include more than one removable drug polymeric drug delivery panel 12 which, in turn, illustrates the ability to customize an implantable medical device for treatment of a hollow fluid carrying organ. In this fashion, a treating physician is able to adapt the implantable medical device as needed without the need for ordering or specifying a custom implantable medical device. For example, the treating physician can use the first removable polymeric drug delivery panel 12 to deliver a first bioactive agent and utilize the second removable polymeric drug delivery panel 12A to deliver a second bioactive agent. Moreover, the treating physician can utilize multiple removable polymeric drug delivery panels to provide an increased dosage of a selected bioactive agent to a lesion within a hollow fluid carrying body organ. As a result of being able to adapt the stent 70 to include two or more removable polymeric drug delivery panels 12 and 12A, a treating physician can perform an emergency procedure on a patient without concerns for the hospital pharmacy having a particular drug coated stent. Those skilled in the art will recognize that the coupling of two or more removable polymeric drug delivery panels 12 to stent 70 is exemplary, and that other implantable medical devices can have coupled thereto more than one removable polymeric drug delivery panels.

FIG. 3 also illustrates a deformable stent structure 25 for use as a pliable element to further secure the removable polymeric drug delivery panel 12 and 12A to a surface of the stent 70. In this manner, once the removable polymeric drug delivery panel 12 or 12A is electrostatically coupled to a surface of the stent 70, the deformable strut structure 25 can be deformed so that each deformable strut structure contacts at least a surface of each of the removable polymeric drug delivery panels 12 and 12A to further secure the panels to the stent 70. The use of the deformable strut structure 25 helps to ensure that the removable polymeric drug delivery panel 12 and 12A remain securely fastened to the stent 70 as the stent is transported through a hollow fluid carrying organ and through the lesion to the treatment site.

FIG. 4 illustrates that the stent 70 is configurable to include two or more removable polymeric drug delivery panels 12, 12A and 12B of variable length. In this manner, the treating physician is able to use the removable polymeric drug delivery panel 12 as a dosemetric control device. As such, the treating physician selects a desired length of the removable polymeric drug delivery panel 12 that contains a desired amount of a selected bioactive agent for use in treating a patient. Those skilled in the art will recognize that the length of the removable polymeric drug delivery panel 12, 12A and 12B is based on a number of factors. Such factors include, but are not limited to, a thickness dimension of the removable polymeric drug delivery panel, a width dimension of the removable polymeric drug delivery panel, an absorbability factor of the removable polymeric drug delivery panel that is based in part on a porosity of the panel, a solubility of the selected bioactive agent, and a method for infusing or compounding the removable polymeric drug delivery panel 12, 12A and 12B with the selective bioactive agent.

FIG. 5 illustrates a stent 70 coupled to a balloon catheter 72, the stent having coupled thereto the removable polymeric drug delivery panel 12. In practice, the stent 70 is secured to the balloon catheter 72 in at least one of a number of suitable manners, such as crimping. FIG. 5 further illustrates the ability to adapt the removable polymeric drug delivery panel 12 to a wide variety of implantable medical device or combination of such devices.

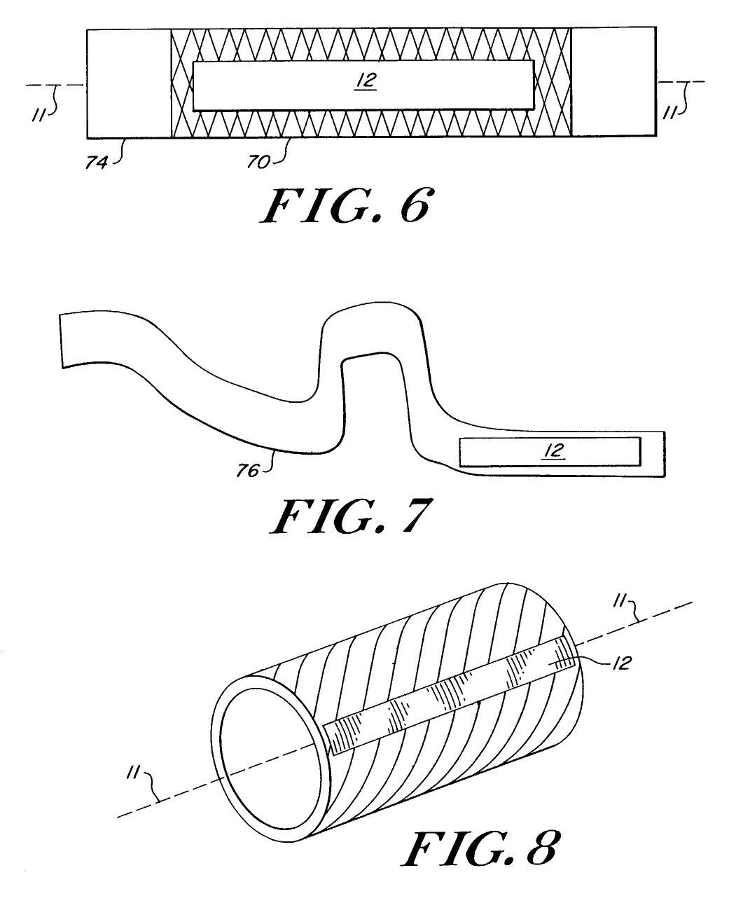

FIG. 6 illustrates a luminal stent graft 74 having a stent member 70 and the removable polymeric drug delivery panel 12. The removable polymeric drug delivery panel 12 extends along the central longitundal axis 11 of the luminal stent graft 74. In this manner, the luminal stent graft 74 is able to kinetically deliver one or more bioactive agents held by the microporous structure of the removable polymeric drug delivery panel 12 over a significant longitundal section of a treatment site within a hollow fluid carrying organ.

FIG. 7 illustrates a catheter 76 having coupled thereto the removable polymeric drug delivery panel 12. FIG. 7 further illustrates the versatility of the removable polymeric drug delivery panel 12 and its advantageous electrostatic coupling so that a number of implantable medical devices can be utilized to advantageously deliver one or more bioactive agents over a significant longitundal section of a treatment site. The catheter 76 as adapted with the removable polymeric drug delivery panel 12 is suitable for treatment of urological disorders or like disorders that typically use a catheter structure as a diagnostic or treatment tool.

FIG. 8 illustrates a vascular graft 78 to which is coupled the removable polymeric drug delivery panel 12 along its longitudinal axis 11. The removable polymeric drug delivery panel 12 is electrostatically coupled to at least a portion of the vascular graft 78. In this manner, the vascular graft 78 can be adapted to include the removable polymeric drug delivery panel 12 just prior to vascular surgery so that the surgeon can advantageously administer one or more bioactive agents as part of the surgical repair of the vascular member. In this way, the surgeon can utilize the removable polymeric drug delivery panel 12 to administer an antibiotic agent directly at the operative site or utilize the removable polymeric drug delivery panel 12 to administer one or more thrombolytic agents or both.

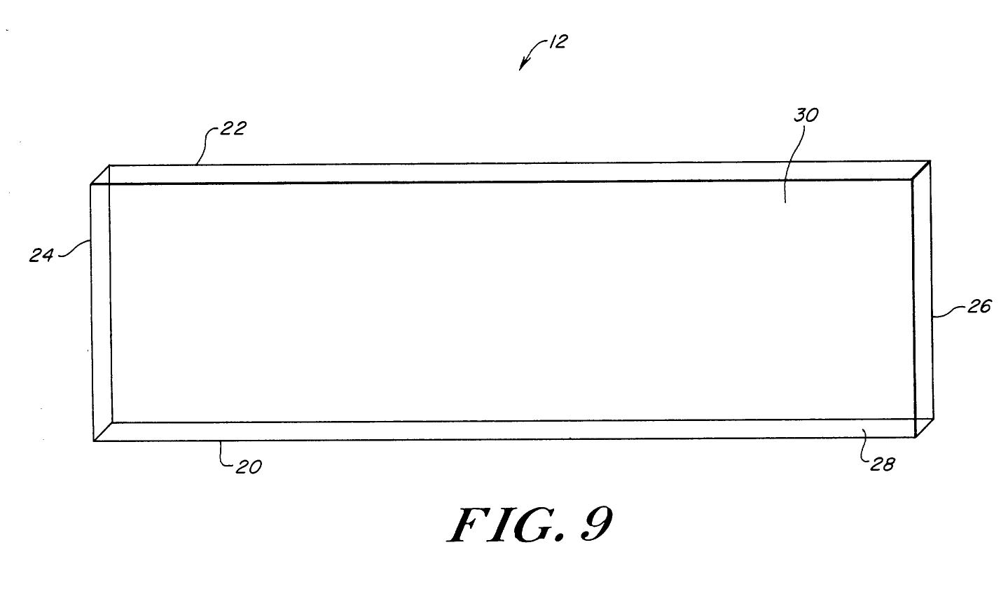

FIG. 9 illustrates a three-dimensional geometric form of the removable polymeric drug delivery panel 12. The geometric form is characterized as a polyhedron having substantially straight and flat surfaces. As illustrated, the removable polymeric drug delivery panel 12 includes a first face surface 28 and second face surface 30. The first and second face surfaces 28 and 30 exhibit a rectangular shape and are in edge contact with a first edge surface 20, a second edge surface 22, a third edge surface 24 and a fourth edge surface 26. The first and second face surfaces 28 and 30 are considered interchangeable in that either the first or second face surfaces 28 and 30 can be coupled to a portion of an outer surface of the radially expandable structure 10.

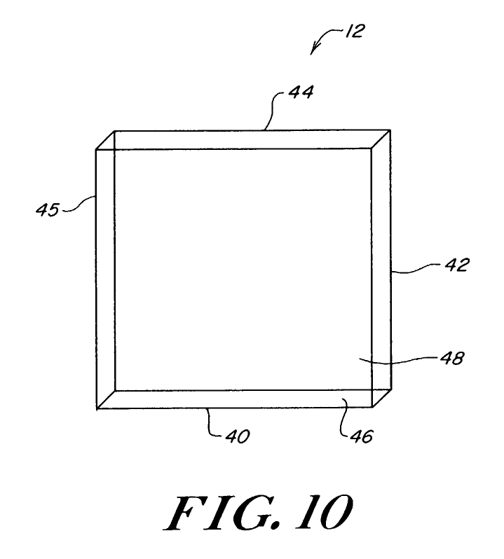

FIG. 10 further illustrates the exemplary removable polymeric drug delivery panel 12 in an alternative embodiment that is suitable for use in the illustrative embodiment of the present invention. The removable polymeric drug delivery panel 12 of FIG. 10 is configured as a three-dimensional geometric form bounded by substantially straight flat surfaces. The geometric form illustrated in FIG. 4 is a polyhedron having a first face surface 48 and a second face surface 46 having a square shape. Those skilled in the art will recognize that either a first face surface 46 or second face surface 48 are suitable for electrostatic coupling with a portion of the outer surface of the radially expandable structure 10 to leave the outer surface for contact with a lumen surface of a hollow fluid carrying body organ. The first face surface 46 and the second face surface 48 are each in contact with and bounded at their edges by a first edge surface 40, a second edge surface 42, a third edge surface 44 and a fourth edge surface 45. Each edge surface 40, 42, 44, 45 being substantially straight and flat, and of uniform thickness.

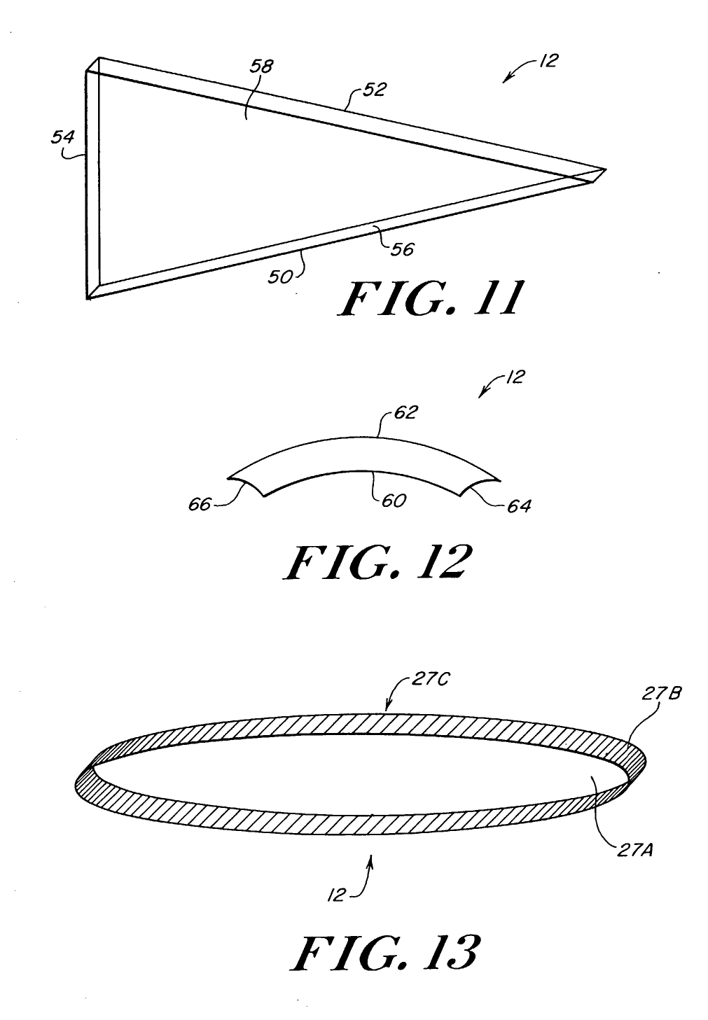

FIG. 11 illustrates a further embodiment of the exemplary removable polymeric drug delivery panel 12 that is suitable for use in the illustrative embodiment of the present invention. The removable polymeric drug delivery panel 12 illustrated in FIG. 11 is characterized as a closed three-dimensional form bounded by substantially straight and flat surfaces to form a tapered polyhedron. The removable polymeric drug delivery panel 12 includes a first face surface 56 and a second face surface 58. Each of the face surfaces 56 and 58 are suitable for contacting either the inner surface of a hollow fluid carrying organ or a portion of the outer surface of the radially expandable structure 10. The removable polymeric drug delivery panel 12 also includes a first edge surface 50, a second edge surface 52 and a third edge surface 54 that contact the edges of the first face surface 56 and the second face surface 58 to form a polyhedron having a gradual dimension in width from a first end portion to a second end portion.

FIG. 12 illustrates the removable polymeric drug delivery panel 12 configured as a closed three-dimensional geometric form bounded by continuous linear arcuate surfaces. As configured, this exemplary embodiment of the removable polymeric drug delivery panel 12 includes a first arcuate face surface 60 and a second arcuate face surface 62. Each arcuate face surface 60, 62 is contourable to a portion of the outer surface of the radially expandable structure 10 in either the first dimension 16 or the second dimension 18. Moreover, each arcuate face surface 60, 62 are also contourable to the curvature and topology of the inner lumen surface of the hollow fluid carrying organ in which the implantable medical device is deployed. The removable polymeric drug delivery panel 12 also includes a continuous linear arcuate edge surface 62 and a second continuous linear arcuate edge surface 64 to bound the three-dimensional geometric shape of the removable polymeric drug delivery panel 12.

FIG. 13 illustrates the removable polymeric drug delivery panel 12 configured as a closed three-dimensional geometric form having an elliptical shape. As configured, this exemplary embodiment of the removable polymeric drug delivery panel 12 includes a first elliptical face surface 27A and a second elliptical face surface 27C. Either the first elliptical face surface 27A or the second elliptical face surface 27C are suitable for electrostatic coupling with a portion of the outer surface of the radially expandable structure 10. The first elliptical face surface 27A and the second elliptical face surface 27C are each in contact with and bounded at their edges by an elliptical edge surface 27B.

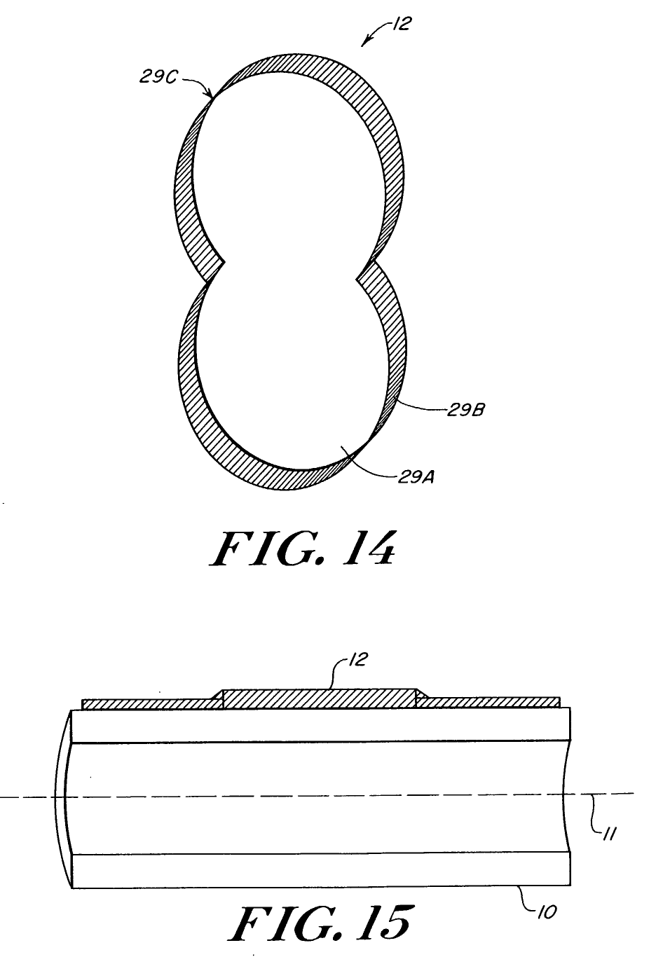

FIG. 14 illustrates the removable polymeric drug delivery panel 12 configured as a closed three-dimensional geometric form bounded by actuate surfaces. As configured, this exemplary embodiment of the removable polymeric drug delivery panel 12 includes a first actuate face surface 29A and a second actuate face surface 29C. Each actuate face surface 29A, 29C is contourable to a portion of the outer surface of the radially expandable structure 10 in either the first dimension 16 or the second dimension 18. Nonetheless, the removable polymeric drug delivery panel 12 illustrated in FIG. 14 is suitable for use with implantable medical devices that have a fixed radial dimension and do not expand from a first dimension to a second dimension as illustrated and discussed above with reference to FIGS. 7 and 8. The removable polymeric drug delivery panel 12 also includes a continuous linear accurate edge surface 29B that bounds the three-dimensional geometric shape of the removable polymeric drug delivery panel 12 illustrated in FIG. 14.

FIG. 15 illustrates the removable polymeric drug delivery panel 12 configured to have a variable thickness dimension. As illustrated, the removable polymeric drug delivery panel 12 is electrostatically coupled to an outer surface to the radially expandable structure 10. Nevertheless, those skilled in the art will recognize that the removable polymeric drug delivery panel 12 having a varying thickness is also suitable for use with the implantable medical devices that are not radially expandable, such as the implantable devices discussed above in relation to FIGS. 7 and 8. Moreover, those skilled in the art will recognize that the removable polymeric drug delivery panel 12 is configurable to have more than two thickness dimensions. Moreover, the removable polymeric drug delivery panel 12 can be adapted at a first end portion or a second end portion having differing thickness or adapted to have multiple thickness dimensions along a longitudinal length of the removable polymeric drug delivery panel 12. Those skilled in the art will recognize that the removable drug delivery panel 12 can be adapted to have a thickness portion that can match a length of a lesion, for example, or can be adapted with a varying thickness dimension so that only a portion of the panel need be loaded with a selected bioactive agent or so that a portion of the panel can be compounded with an increased volume of a selected bioactive agent. Furthermore, the removable polymeric drug delivery panel 12 can have a tapered thickness dimension so that the thickness changes from a first end portion to a second end portion of the removable polymeric drug delivery panel 12 to facilitate insertion of an implantable medical device into a lesion.

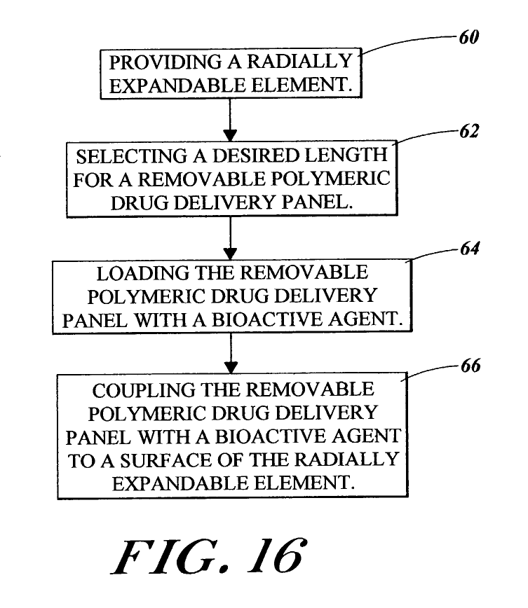

FIG. 16 illustrates a method for manufacturing an illustrative implantable medical device of the present invention. The radially expandable element 10 is provided having a predetermined size and shape based on the size of hollow fluid carrying body organ to receive treatment (step 60). The physician treating the hollow fluid carrying organ selects a desired length of the removable polymeric drug delivery panel 12 for a desired dosage of the selected one or more bioactive agents utilized in the treatment protocol (step 62). The selecting of a desired length of the removable polymeric drug delivery panel 12 advantageously allows the physician to accurately control dosage of the one or more selected bioactive agents. Those skilled in the art will recognize that dosemetric control is based in part on the microporous structure of the removable polymeric drug delivery panel 12, which provides a further benefit of helping to avoid overdosage situations due to the inherent saturation limit of the removable polymeric drug delivery panel 12. Moreover, the removable polymeric drug delivery panel 12 provides for an immediate and linear release of the one or more selected bioactive agents at the treatment site unlike other implantable medical devices having a coated bioactive agent where a protective layer over the bioactive agent must first be absorbed or penetrated before the benefits of the bioactive agent can be realized.

Upon selection of the desired length for the removable polymeric drug delivery panel 12, the physician or other qualified person loads the removable polymeric drug delivery panel 12 with one or more selected bioactive agents (step 64). Once loaded, the removable polymeric drug delivery panel 12 is electrostatically coupled to a portion of an outer surface of the radially expanded element 10 along its longitudinal axis 11 for deployment within a selected hollow fluid carrying organ (step 66). Those skilled in the art will recognize that the physician can also utilize one or more mechanical fasteners or outer fasting techniques such as crimping, to ensure that the removable polymeric drug delivery panel 12 remains securely affixed during travel through the hollow fluid carrying organ and insertion through a lesion. Suitable mechanical fastener means include one or more flexible loop elements such as a suture secured to one or more struts of the radially expandable element 10 or a bendable or flexible strut element capable of pinching a portion of the removable polymeric drug delivery panel 12 to a portion of the radially expandable structure 10. Moreover, those skilled in the art will recognize that the use of a bonding agent to bond a portion of the removable polymeric drug delivery panel to a portion of the radially expandable element is not desirable for the bonding agent or bonding substance inhibits the mobility, transportability, flexibility and deployability of the implantable medical device 10.

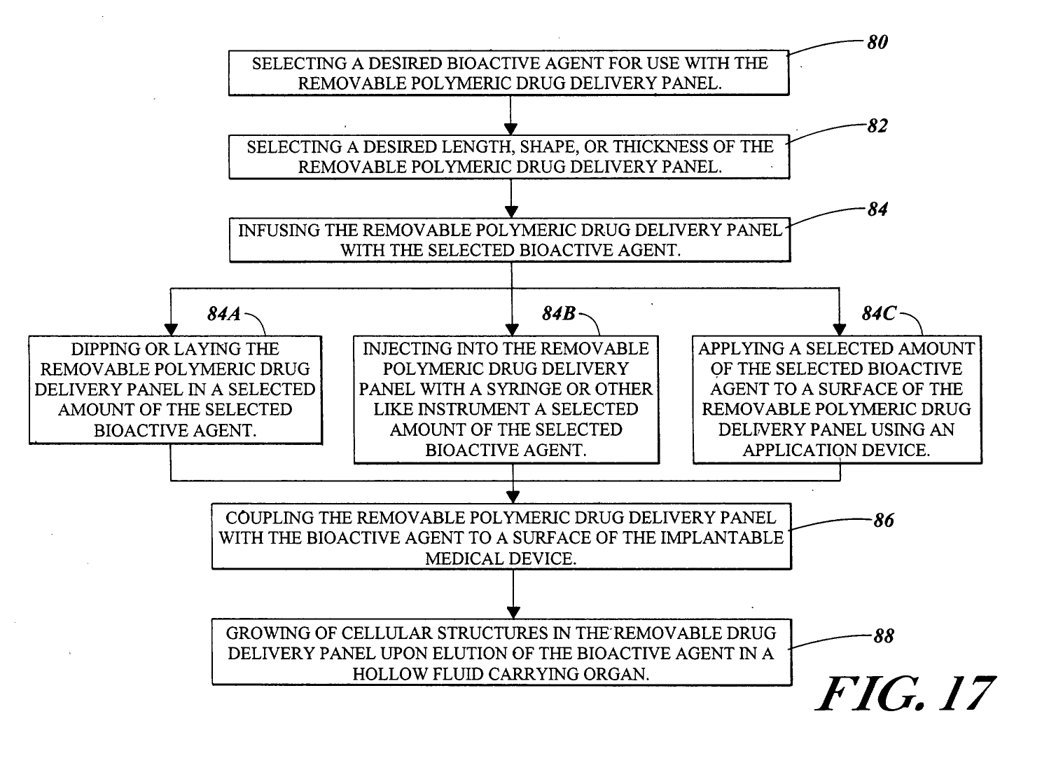

FIG. 17 illustrates one or more steps that a medical professional can utilize to infuse, load or compound the removable polymeric drug delivery panel 12 with a selected bioactive agent. Based on treatment protocol, or other treatment factors, the medical professional, such as the treating physician, pharmacist or other like professional, selects a desired bioactive agent for use with the removable polymeric drug delivery panel 12 (step 80). Having selected the desired bioactive agent, the physician also selects a length and possibly a shape and a thickness of the removable polymeric drug delivery panel 12 to receive the selected bioactive agent (step 82). Those skilled in the art will recognize that the selection of a desired length, shape, thickness, or a number of panels is based on a number of factors that include, but are not limited to, treatment protocol, selected bioactive agent or agents, size of lesion, age of patient and other like factors. Moreover, those skilled in the art will recognize that the order in which the bioactive agent and the removable polymeric drug delivery panel 12 are selected is merely illustrative in that the selection order may be reversed or performed in parallel. Having selected the desired bioactive agent and the desired removable polymeric drug delivery panel 12 the treating physician or other medical professional, such as a nurse or pharmacist infuses the removable polymeric drug delivery panel 12 with the selected bioactive agent (step 84).

The treating physician or other medical professional responsible for infusing the removable drug delivery panel 12 with the selected bioactive agent is able to do so using a number of techniques. In one manner, the responsible medical professional dips or lays the removable polymeric drug delivery panel 12 in a selected amount of the selected bioactive agent until the removable polymeric drug delivery panel 12 has absorbed a sufficient amount of the bioactive agent (step 84A). In another exemplary manner for infusing the removal polymeric drug delivery panel 12 with the selected bioactive agent, the responsible medical professional injects, with a syringe or other like instruments, a selected amount of the bioactive agent into the removal polymeric drug delivery panel 12 (step 84B). In yet another exemplary manner for infusing the removable polymeric drug delivery panel 12 with the selected bioactive agent, the responsible medical professional applies a selected amount of the bioactive agent to a surface of the removal polymeric drug delivery panel 12 using an application device, such as a specialized dosemetric controlled device having a felt tip or rollerball type tip that delivers a predetermined amount of the selected bioactive agent (step 84C). Yet another exemplary manner for infusing the removable polymeric drug delivery panel 12 with the selected bioactive agent, includes instances where the responsible medical professional injects the selected bioactive agent into the removable polymeric drug delivery panel 12 via transcatheter balloon irrigation after the device is deployed into the fluid containing organ.

Those skilled in the art will recognize that other suitable techniques are available for infusing the removable polymeric drug delivery panel 12 with a selected bioactive agent, for example, the removable polymeric drug delivery panel 12 can be purchased with an infused amount of a selected bioactive agent from a manufacturer, such as a pharmaceutical manufacturer that infuses a bioactive into the removable polymeric drug delivery panel 12 during the manufacturing process of the panel or that the responsible medical professional can infuse more than one selected bioactive agent into the removable polymeric drug delivery panel 12. Once the removable polymeric drug delivery panel 12 is infused with the selected bioactive agent, the treating physician, or responsible medical personnel couples the removable polymeric drug delivery panel 12 to a surface of the implantable medical device for treatment of a selected region within the patient (step 86). Upon elution of a portion of the selected bioactive agent from the removable polymeric drug delivery panel 12, the removable polymeric drug delivery panel 12 is capable of supporting cellular growth in the microporous areas that eluted the bioactive agent to stabilize or secure the removable polymeric drug delivery panel 12, the implanted medical device or both to an inner wall of the hollow fluid carrying organ (step 88).

While the present invention has been described with reference to a preferred embodiment thereof, one of ordinary skill in the art will appreciate that various changes in form and detail may be made without departing from the intended scope of the present invention as defined in the pending claims. For example, the implantable medical device may include or be coupled to a delivery device such as balloon catheter. Moreover, those skilled in the art will recognize that more than one bioactive agent can be infused or compounded into the removable polymeric drug delivery panel to facilitate treatment of a patient. Furthermore, those skilled in the art will recognize that the one or more bioactive agents can be selected from one or more immunosuppressive or chemotherapeutic agents such as Paclitaxel, Taxane, Rapamycin, Mycophenolic acid or any derivatives thereof.

What is claimed is:

1. An implantable medical device comprising,
a radially expandable structure with a central longitudinal axis and an outer surface; and
a removable polymeric drug delivery panel electrostatically coupled in a temporary manner to a portion of the radially expandable structure, the removable polymeric drug delivery panel extending along the central longitudinal axis of the radially expandable structure from a first end portion to a second end portion such that a substantial portion of the outer surface of the radially expandable structure following deployment within a fluid containing organ or space is free of the drug delivery panel following deployment.

2. The implantable medical device of claim 1, wherein the removable polymeric drug delivery panel further comprises,
a first contourable surface, the first contourable surface having an electrostatic charge potential and adaptive to a portion of a curvature of the outer surface of the radially expandable structure without substantially limiting uniform expansion of the radially expandable structure.

3. The implantable medical device of claim 1, wherein the removable polymeric drug delivery panel further comprises,
a first contourable surface, the first contourable surface having an electrostatic charge potential and adaptive to a portion of a curvature of the outer surface of the radially expandable structure without substantially inducing said radially expandable structure to recoil to a previous position.

4. The implantable medical device of claim 1, wherein the panel further comprising a thickness dimension substantially less than a width dimension and a length dimension.

5. The implantable medical device of claim 2, wherein the removable polymeric drug delivery panel further comprises at least one bioactive substance releasably introduced into the removable polymeric drug delivery panel for release at a desired location following deployment within a hollow organ by expansion of the radially expandable structure at the desired treatment location in the hollow organ.

6. The implantable medical device of claim 1, further comprising a radially expanding device that temporarily fastens a portion of the removable polymeric drug delivery panel to the outer surface of the radially expandable structure.

7. The implantable medical device of claim 1, wherein the removable polymeric drug delivery panel further comprises a microporous structure of nodes and fibrils, said fibrils having a diameter suitable for cellular fluid communication between two or more cells in said removable polymeric drug delivery panel at the desired treatment location in the fluid carrying organ.

8. The implantable medical device of claim 1, wherein the removable polymeric drug delivery panel further comprises, a microporous structure of nodes and fibrils, said fibrils having a diameter suitable for cellular ingrowth in said removable polymeric drug delivery panel at the desired treatment location in the fluid carrying organ.

9. The implantable medical device of claim 8, wherein the diameter of said fibril is between about 10 Angstroms and about 150 Angstroms.

10. The implantable medical device of claim 8, wherein a first portion of said removable polymeric drug delivery panel is adapted for said cellular ingrowth and a second portion of said removable polymeric drug delivery panel is adapted to inhibit said cellular ingrowth in said second portion.

11. The implantable medical device of claim 10, wherein said first portion and said second portion of said removable polymeric drug delivery panel define a ratio that represents a surface area percentage of said removable polymeric drug delivery panel that is adapted for said cellular ingrowth at the desired treatment location in the fluid carrying organ.

12. The implantable medical device of claim 11, wherein the fastener means comprises a deformable portion of the radially expandable structure.

13. The implantable medical device of claim 6, further comprising a radially expanding device that temporarily fastens a portion of the removable polymeric drug delivery panel to a portion of an outer surface of a deployment delivery catheter.

14. The implantable medical device of claim 6, wherein a portion of the radially expanding device comprises a fastener means.

15. The implantable medical device of claim 14, wherein the fastener means comprises a portion of a deformable radially expandable loop made part of the radially expandable structure before and after crimping onto the deployment delivery catheter.

16. The implantable medical device of claim 15, wherein a portion of the deformable radially expandable loop structure comprises a bendable element made part of the radially expandable structure to bend from a first position to a second position and alternately back to the first position in order to fasten a portion of the removable polymeric drug delivery panel to the outer surface of the radially expandable structure.

17. The implantable medical device of claim 15, wherein a portion of the deformable radially expandable loop structure comprises a bendable element made part of the radially expandable structure to bend from a first position to a second position and alternately back to the first position in order to fasten a portion of the removable polymeric drug delivery panel to the outer surface of the radially expandable structure and to a portion of a deployment delivery catheter.

18. The implantable medical device of claim 1, wherein the removable polymeric drug delivery panel comprises a microporous polymer.

19. The implantable medical device of claim 1, wherein the removable polymeric drug delivery panel comprises a highly electronegative microporous polymer.

20. The implantable medical device of claim 18, wherein the microporous polymer comprises expanded polytetrafluoroethylene (ePTFE).

21. The implantable medical device of claim 5, wherein the removable polymeric drug delivery panel further comprises a second contourable surface adaptive to the curvature of the outer surface of the radially expandable structure and a topology of an inner portion of the hollow organ space made by the radially expandable structure so that a substantial portion of the second contourable surface contacts the inner portion of the hollow organ surface upon expansion of the radially expandable structure to an expanded diameter for kinetic release of the pharmacological compound into the body.

22. The implantable medical device of claim 2, wherein the removable polymeric drug delivery panel further comprises a closed three dimensional geometric form bounded by substantially straight surfaces.

23. The implantable medical device of claim 2, wherein the removable polymeric drug delivery panel further comprises a closed three dimensional geometric form bounded by continuous linear arcuate edge surfaces.

24. The implantable medical device of claim 22, wherein the closed three dimensional geometric form comprises a polyhedron having a first and second surface faces, and a first and second continuous edge surfaces, with each of the faces of the polyhedron having a rectangular shape.

25. The implantable medical device of claim 22, wherein the closed three dimensional geometric form comprises a polyhedron having a first and second surface faces, and a first and second continuous edge surfaces, with each of the faces of the polyhedron having a square shape.

26. The implantable medical device of claim 22, wherein the closed three dimensional geometric form comprises a tapered polyhedron having a first and second surface faces, and a first and second continuous edge surfaces, wherein each of the faces exhibits a gradual diminution in width from a first end portion to a second end portion.

27. The implantable medical device of claim 20, wherein the polyhedron further comprises a substantially uniform thickness throughout.

28. The implantable medical device of claim 24, wherein the polyhedron further comprises a non-uniform thickness throughout.

29. The implantable medical device of claim 25, wherein the polyhedron further comprises a substantially uniform thickness throughout.

30. The implantable medical device of claim 25, wherein the polyhedron further comprises a non-uniform thickness throughout.

31. The implantable medical device of claim 26, wherein the polyhedron further comprises a substantially uniform thickness throughout.

32. The implantable medical device of claim 26, wherein the polyhedron further comprises a non-uniform thickness throughout.

33. The implantable medical device of claim 1, wherein the implantable medical device comprises a device selected from one of, a stent, a balloon catheter, a catheter and an endoluminal stent graft.

34. The implantable medical device of claim 5, wherein a dosage amount of the at least one bioactive substance held by said removable polymeric drug delivery panel is not limited by an outer surface area of the radially expandable structure.

35. The implantable medical device of claim 5, wherein the release of the at least one bioactive substance held by said removable polymeric drug delivery panel at the desired location following deployment is not dependent upon a breakdown of the removable polymeric drug delivery panel following deployment at a treatment site.

36. A method for manufacturing an expandable implantable medical device, the method comprising the steps of,
providing a radially expandable element having a central longitudinal axis and an outer surface; and
electrostatically coupling a removable polymeric drug delivery element to at least a portion of the outer surface of the radially expandable element along the central longitudinal axis so that the removable polymeric drug delivery element extends along the central longitudinal axis from a first end portion to a second end portion of the radially expandable element to cover a portion of the outer surface along the longitudinal axis from the first end portion to the second end portion and to leave a remaining portion of the outer surface of the radially expandable element free of the removable polymeric drug delivery element.

37. The method of claim 36, further comprising the step of, attaching a fastener element to the radially expandable element to allow a portion of the removable polymeric drug delivery element to be mechanically fastened to a portion of the outer surface of the radially expandable element.

38. The method of claim 37, wherein the therapeutic amount of the selected bioactive agent held by said removable polymeric drug delivery panel is not limited by a surface area of the outer surface of the radially expandable element.

39. The method of claim 37, wherein the local administration of the selected bioactive agent held by said removable polymeric drug delivery panel at the selected site within the hollow organ space occurs without a portion of said removable polymeric drug delivery element dissolving within the hollow organ space.

40. The method of claim 37, further comprising the step of, attaching a fastener element to the radially expandable element to allow a portion of the removable polymeric drug delivery element to be mechanically fastened to a portion of the outer surface of the radially expandable element and a deployment delivery catheter.

41. The method of claim 36, further comprising the step of, loading the removable polymeric drug delivery element with a therapeutic amount of a selected bioactive agent to locally administer the selected bioactive agent at a selected site within a hollow organ space made by the radially expandable element in the body.

42. The method of claim 36, further comprising the steps of,
infusing the removable polymeric drug delivery element with at least one pharmaceutical compound;
selecting a desired length of the removable polymeric drug delivery element; and
cutting the removable polymeric drug delivery element to the selected desired length in order to select a dosemetric controllable means of the bioactive agent for local administration of the bioactive agent at a selected site within a hollow organ space made by the radially expandable element in the body.

43. The method of claim 37, wherein the fastener comprises a portion of an expanded PTFE flat film formed into a loop so that a portion of the radially expandable element can be passed through at least a portion of the expanded PTFE flat film loop to mechanically fasten the removable polymeric drug delivery element to the outer surface of the radially expandable element and a deployment delivery catheter.

44. The method of claim 37, wherein the fastener comprises a pliant element affixed to the radially expandable structure, the pliant element being pliable and adjustable from a first position to a second position and alternately back to the first position in order to fasten a portion of the removable polymeric drug delivery panel to the outer surface of the radially expandable structure.

45. The method of claim 44, further comprising the step of,
growing said cellular structure in a first portion of the removable polymeric drug upon elution of the selected bioactive agent at the selected site within the hollow organ space; and
inhibiting said cellular structure from growing in a second portion of the removable polymeric drug upon elution of the selected bioactive agent at the selected site within the hollow organ space.

46. The method of claim 36, wherein the removable polymeric drug delivery element comprises a closed three-dimensional geometric shape bounded by substantially straight surfaces.

47. The method of claim 37, further comprising the steps of, growing a cellular structure in the removable polymeric drug delivery element upon elution of the selected bioactive agent at the selected site within the hollow organ space.

48. The method of claim 46, wherein the closed three dimensional geometric shape comprises a polyhedron having a first and second face surfaces, and a first and second continuous edge surfaces, with each of the faces of the polyhedron having a rectangular shape.

49. The method of claim 46, wherein the closed three dimensional geometric shape comprises a polyhedron having a first and second face surfaces, and a first and second continuous edge surfaces, with each of the faces of the polyhedron having a square shape.

50. The method of claim 46, wherein the closed three dimensional geometric shape comprises a tapered polyhedron having a first and second face surfaces, and a first and second continuous edge surfaces, wherein each of the faces exhibits a gradual diminution in width from a first end portion to a second end portion.

51. The method of claim 36, wherein the expandable element comprises an element selected from one of a stent, a graft, a balloon stent and a self expanding stent catheter.

52. The method of claim 36, wherein the removable polymeric drug delivery element comprises a microporous material having at least one substantially flat surface.

53. The method of claim 52, wherein the microporous material comprises expanded polytetrafluoroethylene (ePTFE).

54. A stent for hollow organ tissue contact and bioactive drug delivery comprising,
an expandable tubular element having an inner passage, a longitudinal axis and an outer wall,
at least one removable microporous polymeric panel element electrostatically coupled to at least a portion of the outer wall of the expandable tubular element along the longitudinal axis from a distal portion to a proximal portion of the expandable tubular element so that the outer surface of the expandable tubular element includes a longitudinally porous surface contact portion, the panel having at least a first surface profile and a second surface profile with each surface profile having a curvature substantially matching a surface profile to that of an arcuate outer wall shape of the outer wall of the expandable tubular element before and after deployment within the hollow organ targeted for therapeutic treatment, and
a bioactive agent compounded into the microporous polymeric element, wherein upon expansion of the expandable tubular element at least one surface of the microporous polymeric panel element maintains continuous direct contact with an inner wall surface of a selected hollow organ within the body.

55. The stent of claim 54, further comprising a portion of a pliable element to affix a portion of the microporous polymeric panel element to the expandable tubular element, the pliable element adapted to flex from a first position to a second position and back to the first position to allow a portion of the microporous polymeric panel element to be looped through the pliable element or around the pliable element to affix the portion of the microporous polymeric panel element to the expandable tubular element.

56. The stent of claim 54, wherein the pliable element comprises a structure selected from one of a loop structure, a hook structure and a deformable strut structure.

57. The stent of claim 54, wherein the microporous polymeric panel element comprises a closed three dimensional geometric shape bounded by substantially straight surfaces, the surfaces adaptive to include linear arcuate shape surfaces.

58. The stent of claim 54, wherein an amount of the bioactive agent compounded into the microporous polymeric element is not limited by a strut surface area of said stent.

59. The stent of claim 55, wherein the wherein the closed three dimensional geometric shape comprises a polyhedron having a first and second face surfaces, and a first and second continuous edge surfaces, with each of the face surfaces of the polyhedron having a rectangular shape.

60. The stent of claim 57, wherein the closed three dimensional geometric shape comprises a polyhedron having a first and second face surfaces, and a first and second continuous edge surfaces, with each of the face surfaces of the polyhedron having a square shape.

61. The stent of claim 57, wherein the closed three dimensional geometric shape comprises a tapered polyhedron having a first and second face surfaces, and a first and second continuous edge surfaces, wherein each of the face surfaces exhibit a gradual diminution in width from a first end portion to a second end portion.

62. The stent of claim 54, wherein the microporous polymeric panel element comprises expanded polytetrafluoroethylene (ePTFE).

63. A medical device for administering a bioactive substance to a location within a fluid containing organ comprising,
a microporous bioactive substance delivery panel attachable to a surface of a structure suitable for delivering the microporous bioactive substance delivery panel to the location within the fluid containing organ, the microporous bioactive substance delivery panel having a plurality of surfaces and a height dimension significantly less than a length dimension and a width dimension.

64. The medical device of claim 63, wherein the microporous bioactive substance delivery panel substantially maintains, the height dimension, the width dimension and the length dimension following elution of the portion of the bioactive substance.

65. The medical device of claim 63, wherein at least one of the plurality of surfaces of the microporous bioactive substance delivery panel comprises an electronegative charge sufficient to at least temporarily attach the microporous bioactive substance delivery panel to a surface of the structure.

66. The medical device of claim 63, wherein the height dimension, the width dimension and the length dimension of the microporous bioactive substance delivery panel comprise a dosage indicator of the bioactive substance.

67. The medical device of claim 63, wherein the microporous bioactive substance delivery panel further comprises expanded polytetrafluoroethylene (ePTFE).

68. The medical device of claim 63, wherein at least one of the plurality of surfaces is contourable to a surface topology of the structure to which it is attachable without impeding operation of the structure within the fluid carrying organ.

69. The medical device of claim 63, wherein the microporous bioactive substance delivery panel is capable of supporting cellular growth of the fluid containing organ in one or more pores of the microporous structure of the microporous bioactive substance delivery panel following elution of a portion of the bioactive substance from the one or more pores of the microporous structure of the microporous bioactive substance delivery panel into the fluid containing organ.

70. A microporous polymeric panel suitable for coupling to a surface of an implantable medical device for delivery of a selected bioactive agent held by the microporous polymeric panel to a lesion in a hollow fluid carrying organ, said microporous polymeric panel comprising:
a first microporous structure suitable for holding said selected bioactive agent and capable of allowing cellular growth in said first microporous structure following elution of a portion of said selected bioactive agent into a portion of said hollow fluid carrying organ, said microporous polymeric panel is secured to said surface of said implantable device in a manner that does not depend on chemical bonding.

71. The microporous polymeric panel of claim 70 further comprising, a second microporous structure suitable for holding said selected bioactive agent and capable of inhibiting cellular growth in said second microporous structure following elution of a portion of said selected bioactive agent into a portion of said hollow fluid carrying organ.

72. The microporous polymeric panel of claim 64, wherein an amount of the selected bioactive agent held by the microporous polymeric panel is based in part on a porosity of the microporous polymeric panel.

73. The microporous polymeric panel of claim 64, wherein said microporous polymeric panel substantially maintains a surface area in contact with the lesion in the hollow fluid carrying organ following elution of said selected bioactive agent.

74. The microporous polymeric panel of claim 71, wherein said second microporous structure holds a second selected bioactive agent and is capable of inhibiting cellular growth in said second microporous structure following elution of a portion of said second bioactive agent into a portion of said hollow fluid carrying organ.

75. The microporous polymeric panel of claim 70, wherein said microporous polymeric panel is electrostatically coupled to said surface of said implantable medical device.

76. The microporous polymeric panel of claim 71 wherein an area of said first microporous structure and an area of said second microporous structure define a surface area ratio of said microporous polymeric panel that indicates a percentage of a surface area of said microporous polymeric panel capable of allowing cellular growth in said microporous polymeric panel following elution of said selected bioactive agent.

77. The microporous polymeric panel of claim 76, wherein said percentage of said surface area of said microporous polymeric panel is based on a total surface area of said microporous polymeric panel.

78. The microporous polymeric panel of claim 76, wherein said percentage of said surface area of said microporous polymeric panel is based on a first surface of said microporous polymeric panel.

79. The microporous polymeric panel of claim 76, wherein said percentage of said surface area has a range of about 0% to about 100% in about increments of 5%.
